(19) United States
(12) Patent Application Publication
(10) Pub. No.: US 2005/0182326 A1
(43) Pub. Date: Aug. 18, 2005

(54) Combined therapy and imaging ultrasound apparatus

(76) Inventor: David Vilkomerson, Princeton, NJ (US)

Correspondence Address: PLEVY & HOWARD, P.C., P.O. BOX 226, FORT WASHINGTON, PA, 19034, US

(21) Appl. No.: 11/057,513

(22) Filed: Feb. 14, 2005

Related U.S. Application Data
(60) Provisional application No. 60/545,301, filed on Feb. 17, 2004.

(51) Int. Cl. A61B 8/00

(52) U.S. Cl. 600/439

(57) ABSTRACT

An ultrasound imaging and treatment system including: a diffraction grating transducer; and, a signal source electrically coupled to the diffraction grating transducer and operative: in a first mode to provide a wide-band excitation signal to the diffraction grating transducer to operate the diffraction grating transducer in an imaging manner; and in a second mode to provide a narrow-band excitation signal to the diffraction grating transducer to operate the diffraction grating transducer in a high intensity focused ultrasound insonifying manner.

### RELATED APPLICATION

This application claims priority of U.S. patent application Ser. No. 60/545,301, entitled COMBINED THERAPY AND IMAGING ULTRASOUND APPARATUS, filed Feb. 17, 2004, the entire disclosure of which is hereby incorporated by reference herein.

### FIELD OF INVENTION

The preset invention relates generally to ultrasonic apparatus and methods, and more particularly to ultrasonic therapeutic hyperthermic procedures and real-time ultrasound volume imaging systems.

### BACKGROUND OF INVENTION

As discussed in a recent review article “High intensity focused ultrasound: surgery of the future?”(British Journal of Radiology, September, 2003; pages 590-599), imaging has been investigated for use in connection with High Intensity Focused Ultrasound (HIFU) to guide the placement of the focused ultrasound energy used to kill undesired tissue. Indeed, HIFU use has dramatically increased in recent years. This article reviews treatment of tumors in the prostate, liver, kidney, breast, bone, uterus and pancreas, for conduction disorders in the heart and even to achieve surgical haemostasis. This article concludes with the finding that “ . . . recent technological development suggests that HIFU is to play a significant role in future surgical practice.”

HIFU systems, such as the SonoBlate 500 from Focus Surgery, of Indianapolis, IN, are commercially available. The United States Food and Drug Administration (FDA) has-cleared HIFU treatment of benign prostate enlargement. Systems for treating prostate cancer are undergoing clinical trials. The SonoBlate 500 includes a mechanically-scanned ultrasound imaging transducer coupled to a HIFU transducer.

Conventional imaging and HIFU systems include mechanically scanned systems, such as those depicted in U.S. Pat. Nos. 6,635,054, (Fjield, et al) and 5,762,066 (Law, et al).

Mechanical systems produce one line of imaging information for each imaging pulse. As each imaging pulse requires some tens of microseconds to propagate and return from the interrogated tissue, generating the thousands of imaging points required for 3-dimensional images is not possible in real-time using these systems. As real-time volumetric imaging is desirable for visualizing targeted tissue, this limitation is significant.

Phased arrays for both imaging and therapy are discussed in Ebbini (“A spherical-section ultrasound phased array applicator for deep localized hyperthermia”, IEEE Trans Biomed Eng, 38, 634-643, 1991) and in U.S. Pat. Nos. 6,613,004 (Vitek et. al) and 6,506,171 (Vitek et. al), for example. U.S. Pat. No. 6,589,174 (Chopra) describes using different frequencies to attain different depths of therapy. U.S. Pat. No. 6,500,121 (Slayton et. al) discloses three-dimensional imaging to guide therapy.

Phased-arrays generally have the capability of generating multiple points per round-trip time, and can accordingly generate real-time volumetric images (Von Ramm et al, “Real-time volumetric imaging system, Parts I & II”, IEEE Trans Ultrasonics, Ferroelectrics, and Frequency Control 1991, 38, 100-115). However, phased array systems require each transducer to be driven independently to achieve the required phasing. This independent driving requirement for each transducer element makes phased array systems undesirably complex. This is particularly true for probes used for insertion into body cavities, as the many cables required for such probes make such systems generally impractical. Since many important applications of HIFU would benefit from the capability to use intra-cavity probes (for treatment of the uterus, bladder, heart, and blood vessels for example), the above-mentioned problems represent a significant obstacle to using phased-array guidance for HIFU.

### SUMMARY OF THE INVENTION

An ultrasonic imaging system comprising: a diffraction grating transducer; and a signal source electrically coupled to the diffraction grating transducer and operative: in a first mode to provide a wide-band excitation signal to the diffraction grating transducer to operate the diffraction grating transducer in an imaging mode; and in a second mode to provide a narrow-band excitation signal to the diffraction grating transducer to operate the diffraction grating transducer in a high intensity focused ultrasound insonifying mode.

### BRIEF DESCRIPTION OF THE DRAWINGS

Understanding of the present invention will be facilitated by consideration of the following detailed description of the preferred embodiments of the present invention taken in conjunction with the accompanying drawings, in which like numerals refer to like parts, and:

FIG. 1 illustrates a schematic representation of a forward-looking, volumetric imaging and HIFU apparatus according to an aspect of the present invention;

FIG. 2 illustrates a manner in which patterns of HIFU treated areas can be achieved by means of the frequency and time of application as a function of angular position, using the apparatus of FIG. 1, according to an aspect of the present invention;

FIG. 3 illustrates a different pattern of HIFU application that may be obtained by using patterns of changing frequency with angular position, using the apparatus of FIG. 1, according to an aspect of the present invention; and

FIGS. 4, 5A and 5B illustrate schematic views of side-looking imaging and HIFU apparatus, and corresponding methods of operation, according to aspects of the present invention.

### DETAILED DESCRIPTION OF THE INVENTION

It is to be understood that the figures and descriptions of the present invention have been simplified to illustrate elements that are relevant for a clear understanding of the present invention, while eliminating, for the purpose of clarity, many other elements found in typical ultrasonic transducer systems and methods of making and using the same. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein.

A system for providing real-time volumetric ultrasound images requiring only two coaxial cables is disclosed in U.S. Pat. No. 6,176,829 ('829 Patent), the entire disclosure of which is hereby incorporated by reference herein. As is described in detail therein, the system includes a diffraction-grating transducer (“DGT”) that produces or receives an ultrasonic beam at an angle directly related to the ultrasound frequency. A coupled lens (or other focusing device, e.g., a mirror) focuses the beam to a position on the lens's focal plane corresponding to the beam's angle. By the linearity of the system, driving and receiving at multiple frequencies simultaneously produces multiple imaging points with each pulse, allowing real-time volumetric imaging when the system is rotated.

A curved DGT may be used without a lens (as it is well known that a curved mirror may be used in place of a lens for focusing), producing focused points for each frequency. Fabricating the DGT on a curved surface achieves such a result. The focusing power of a curved surface can be combined with that of a lens in a combination focusing system for the DGT.

According to an aspect of the present invention, HIFU therapeutic capability may be used in combination with such a volumetric real-time imaging system to provide a system well-suited for clinically significant image-guided therapy in intra-body cavities and vessels.

According to an aspect of the present invention, the volumetric imaging principles of the incorporated '829 Patent may use multiple frequencies to produce multiple beams; while a single frequency (or a limited range of frequencies) may be used to concentrate acoustic energy at a single angle (or limited range of angles) that focus to a single spot (or region) for HIFU therapy.

According to an aspect of the present invention, methods of controlling the frequency and rotation of a single diffraction-grating transducer to control the insonified area for both imaging and therapy may be provided. Further, the use of an additional, but coupled, diffraction-grating-transducer or non-diffracting transducer, if called for by the particular HIFU therapeutic application, may also be provided.

According to an aspect of the present invention, by combining a real-time, volumetric forward- or side-looking ultrasound imaging capability with high-intensity focused ultrasound (“HIFU”) therapy, a system well-suited for image-guided surgery may be provided.

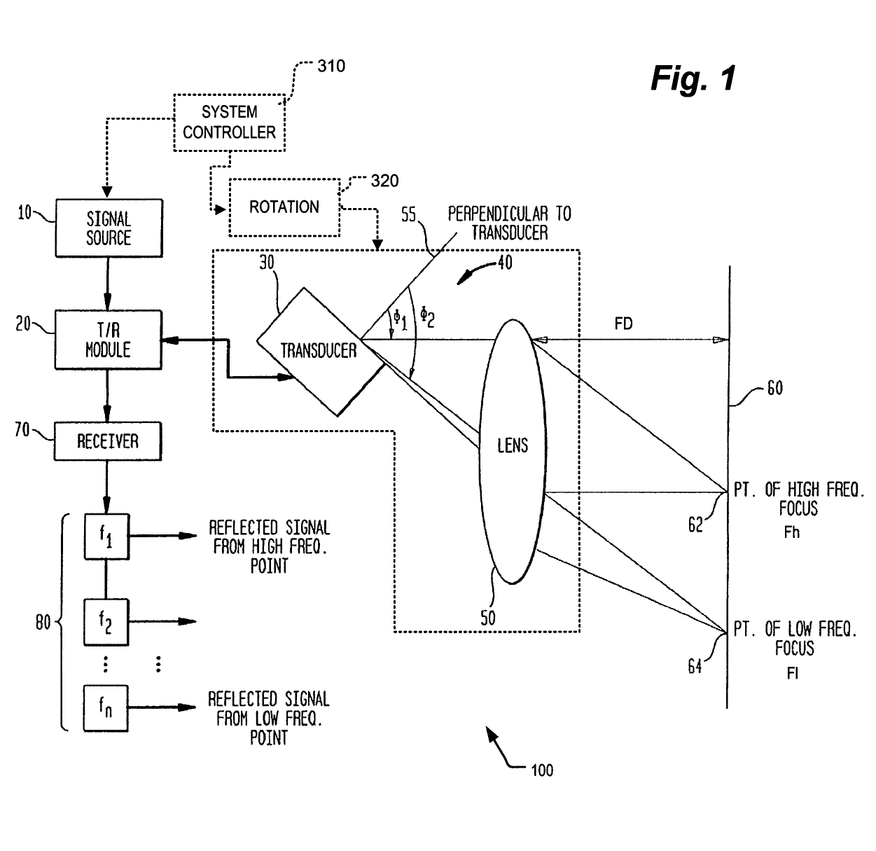

Referring now to the drawings, wherein like references identify like elements of the invention, FIG. 1 illustrates a schematic representation of a forward-looking, volumetric imaging and HIFU system 100 using a single DGT according to an aspect of the present invention. In general, system 100 includes a signal generator 10 for providing a short time duration signal, i.e. a wide-band signal, to transmit receive module (T/R module 20) in an imaging mode. The signal generator or pulse generator 10 produces a series of short time interval pulses at a predetermined pulse rate in an imaging mode of operation. The T/R module 20 is electrically coupled to the pulse generator 10 and transmits each of the pulses to diffraction grating transducer (DGT) 30. DGT 30 produces multiple beams corresponding to multiple frequencies 40 ranging from a high frequency generated at a given angle Φ1 to a low frequency at an angle Φ2 in an imaging mode. As shown in FIG. 1, the angles are measured from a plane 55 perpendicular to DGT 30. In an imaging mode, each of the multiple beams and hence multiple frequencies in the spectral domain, which result from the impulse generated by the pulse generator, impinge at different angles onto lens 50. Focusing lens 50 receives the multiple frequency beams and focuses the different frequency signals to a series of intensity points or spots (fh 62 . . . fl 64) on the back focal plane 60.

The position of the spots on focal plane 60 corresponds to the frequency (i.e., angle) values associated with each of the beams. Each of the focus spots, as is well known through the use of conventional ray tracing, produces reflected signals which travel back through the focusing lens 50 to the quadrature DGT 30 , which delivers the reflected signals to receiver 70. The reflected signals of all the frequencies sent to the receiver 70 are input to a bank of filters 80 (f1, f2, . . . fn), each filter being tuned to a distinct frequency range in order to capture that back scattered frequency associated with the reflected energy from one portion of the focal plane. The output of the filters 80 may be used to drive a conventional display. Because the back-scattered frequency signals can be captured simultaneously using the bank of filters, a line image of the object at the focal plane may be generated. By rotating the transducer 30 and lens 50 at pre-defined angles about an axis in obtaining the reflected signals, a series of lines of points (i.e., like spokes in a wheel) may be generated. By summing the magnitudes of the back scattered frequencies received over the course of the rotation, a three dimensional visualization of an object may be formed.

A controller 310 may be operatively coupled to any conventional apparatus 320 for rotating the transducer 30 and/or lens 50, such as a stepper motor, for example. Such a controller may take the form of a processor, for example. “Processor”, as used herein, refers generally to a computing device including a Central Processing Unit (CPU), such as a microprocessor. A CPU generally includes an arithmetic logic unit (ALU), which performs arithmetic and logical operations, and a control unit, which extracts instructions (e.g., code) from memory and decodes and executes them, calling on the ALU when necessary. “Memory”, as used herein, refers to one or more devices capable of storing data, such as in the form of chips, tapes or disks. Memory may take the form of one or more random-access memory (RAM), read-only memory (ROM), programmable read-only memory (PROM), erasable programmable read-only memory (EPROM), or electrically erasable programmable read-only memory (EEPROM) chips, by way of further non-limiting example only. The memory utilized by the processor may be internal to external to an integrated unit including the processor. For example, in the case of a microprocessor, the memory may be internal or external to the microprocessor itself. Of course, controller 310 may take other forms as well, such as an electronic interface or Application Specific Integrated Circuit (ASIC).

By way of further example, apparatus 100 may image using the following process. DGT 30 produces beams at an angle that depends upon the excitation frequency, as described by:

$$sin ( θ ) = \frac{λ}{d} \qquad ( 1 )$$

where θ is the beam angle, λ is the wavelength of the ultrasound and d is the periodicity of the grating. Lens 50, which is optically coupled to DGT 30, produces a focused spot at a position on the lens's focal plane below the lens axis at a distance equal to:
z=tanθ′FD (2)
where z is the distance, θ′ is the θ given by equation (1) less the “tilt” angle of DGT 30 (set so that the highest frequency of operation of the DGT produces a spot on the lens axis), and FD is the focal distance.

As shown in FIG. 1, if signal source 10 produces a range of frequencies, fh to fl, representing the highest frequency of operation fh to lowest fl, each frequency will produce a focal spot ( 62 . . . 64 ) along a line in the focal plane 60. Ultrasound energy back-scattered from each spot impinges on the lens 50, which directs the energy to DGT 30. By analyzing the amount of energy in each frequency component received by DGT 30, the reflectivity of that spot in space can be mapped. As the system rotates, the line of dots sweeps over an area. If a pulse containing many frequencies is used, the reflected energy at each point can be time-resolved into the reflectivity at a certain distance, so that volume information can be derived, leading to volumetric imaging. A more detailed discussion of imaging with system 100, in general, may be found in the incorporated U.S. Pat. No. 6,176,829.

According to an aspect of the present invention, a single frequency, or discrete set of frequencies, may be provided by source 10 to energize DGT 30, such that the ultrasonic energy will appear and is concentrated at the one point, or discrete set of points, corresponding to that one frequency, or that discrete set of frequencies, in a therapeutic treatment mode. If DGT 30 is driven at a high power, the condition for HIFU, i.e., high energy focused ultrasound may thus be achieved. When several frequencies are applied simultaneously, the power is divided among the spots, and several points can be HIFU insonified simultaneously.

According to an aspect of the present invention, by controlling the excitation frequency, which controls the radial position of the focused ultrasound, and when power is applied to a rotating DGT 30, which controls the angular position θ, any point in the focal plane can be HIFU insonated. By changing the position of the system along the axis of the lens, the HIFU point can be placed in different planes. Thus, according to an aspect of the present invention, HIFU can be placed at any point in the targeted volume, such as in front, of system 100. According to an aspect of the present invention, controller 310 may also serve to selectively operate the transducer 30 in an imaging mode and a HIFU insonifying mode though selective operation of source 10 in wide-band and narrow-band excitation modes.

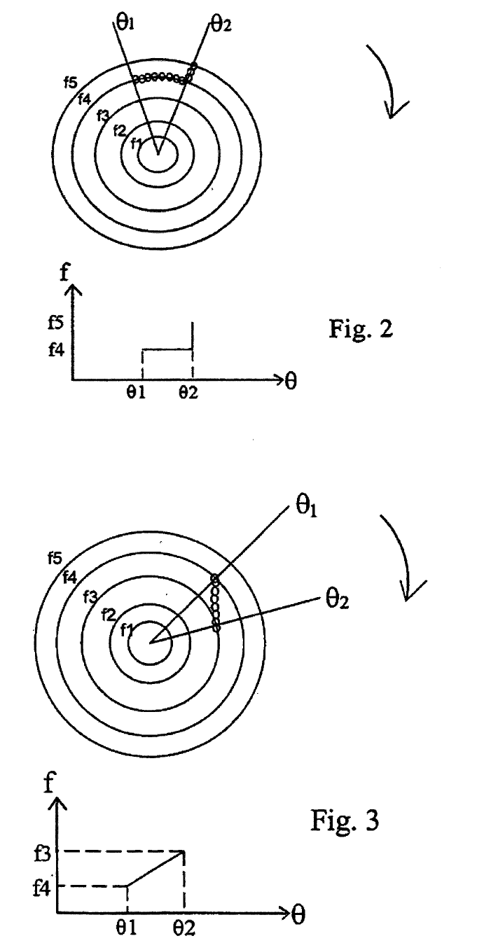

FIGS. 2 and 3 illustrate how patterns of HIFU treated areas can be achieved by means of the frequency and time of application as a function of angular position (FIG. 2); and how a different pattern of HIFU applications can be obtained by using patterns of changing frequency with angular position (FIG. 3). In FIG. 2, pulses at frequency f4 are produced while the system is rotated from θ1 to θ2, producing HIFU points in the focal plane along a circular arc corresponding to the radius for f4. At θ2, simultaneous multiple frequencies (equivalent to a “chord” in music) between f4 and f5 driving the DGT will produce a line of HIFU points along the corresponding θ2 radial line. The driving frequencies as a function of the angular position of the system are also shown in FIG. 2.

In FIG. 3, a straight line of HIFU points can be formed by changing the frequency as a function of the rotational angle. If the frequency increases with the rotational angle, the HIFU spot may trace out the path shown. Again, the driving frequencies as a function of the angular position of the system are also shown in FIG. 3.

Thus, according to an aspect of the present invention, by controlling the frequency or frequencies of excitation, the rotational angle of the system and its position along its axis, any desired pattern of imaging or HIFU insonation in the target volume of the system can be obtained. Further, different diffraction grating transducer excitation frequencies and powers may be sequenced by signal source 10 responsively to controller 310 to provide for substantially simultaneous, real-time imaging and HIFU treatment of a target volume, for example. FIG. 1 shows a target volume in front of the system.

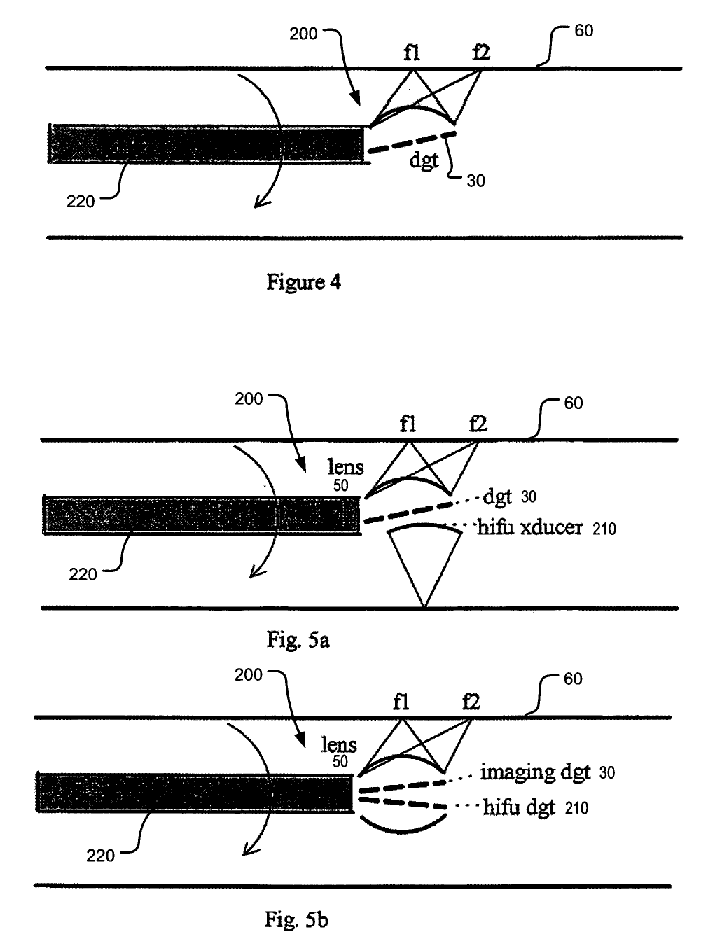

Referring now also to FIG. 4, there is shown a schematic view of a side-looking imaging and HIFU system 200, and a corresponding method of operation, according to aspects of the present invention. Like system 100, system 200 provides for combined therapy and imaging insonification of a target volume. System 200 may be particularly well suited for use with cylindrical shaped objects such as arteries and veins. Cylindrical imaging is appropriate when the images to be obtained, such as plaque on a vessel wall, are themselves substantially cylindrical. As will be understood by those possessing an ordinary skill in the pertinent arts, a forward-looking imaging system would have such wall structures at the edges of their imaging fields, if they are even imaged at all. In contrast, system 200 is well suited for imaging such wall structures.

The electronics for the configuration of FIG. 4 may be analogous to those of FIG. 1. For example, using multiple pulsed frequencies to energize DGT 30, a line of focal spots (fl . . . f2 ) may analogously be formed from the multiple angle beams from DGT 30. By way of further non-limiting example, the highest frequency may be focused at f1, and the lowest frequency at f2. Each spot's back-scattered energy may be received and analyzed by a filter bank analogous to that of FIG. 1, to determine the reflectivity of each spot in the same manner as that discussed in connection with FIG. 1. As the system is rotated and the reflectivity at each frequency as a function of time is analyzed, a volumetric cylindrical image may be generated in real-time.

Similarly, analogously to the configuration shown in FIG. 1, by controlling the frequency or frequencies of excitation as a function of the rotation, a spot or line of spots of HIFU can be generated. For example, if single frequency excitation is used, a circumferential line of HIFU energy may be provided. If multiple frequencies are used at one angle of rotation, a line of HIFU energy may be formed on the vessel wall parallel to its axis. Again, combining selected frequencies and rotation may allow any pattern of HIFU to be generated.

Regardless of specific configuration, for HIFU to heat the tissue to a killing temperature of 50 deg. C., the power must be sufficiently high. As discussed in the paper by C. R. Hill, “Lesion Development in Focused Ultrasound Surgery: A general model”, (Ultrasound in Medicine and Biology, vol 20, pp 259-269), about 50 Joules/gm, if delivered in a short enough time may be therapeutically effective. Resulting heat conduction out of the volume may not be significant when short duration delivery (<few hundred milliseconds) is used. When such conduction is important, the perfusion rate of blood through the target tissue and interface conditions to surrounding tissues become important, as will be understood by those possessing an ordinary skill in the pertinent arts.

The ratio of the focal distance, FD, to the diameter of the lens, known as the f#, determines the beam diameter at the focal plane. For example, if the lens in use is f/3, and the ultrasonic frequency is 15 MHz (λ=0.1 mm), the focused spot region containing >90% of energy can be found from:
Diameter of beam≈F#*λ (3)
as ˜0.3 mm. At 15 MHz, the absorption of ultrasound as it propagates in tissue is ˜1.5 dB/mm, such that 80% of the energy will be absorbed in ˜5 mm of tissue thickness. Therefore, the HIFU power is absorbed in a volume of (π*0.3²*0.25 * 5)˜3.5 10⁻⁴ cm³. The energy required (assuming a short pulse) to produce a killed volume of tissue (assuming tissue density is 1 gram/cm³) is therefore (50*0.8*3.5×10⁻⁴) Joules, or 14 millijoules. If the acoustic power is 10 W (requiring a voltage of ˜140 Vₚ₋ₚ drive to the DGT), a pulse 1.4 milliseconds long will kill the tissue in that volume.

Accordingly, for many therapeutic requirements, HIFU therapy can be accomplished by controlling the rotational speed and the number of simultaneous frequencies (simultaneous focused spots) using a same DGT for both imaging and HIFU according to an aspect of the present invention. However, for some HIFU requirements, for example where large volumes of tissue are to insonified and destroyed, an imaging DGT may be coupled with another transducer. This may overcome the limitation that providing wide bandwidth optimal for DGT imaging usually requires relatively low efficiency in electrical to acoustic power conversion, while an inefficient transducer tends to overheat when high power is applied.

By way of further, non-limiting example only, the cylindrical imaging system 200 of FIG. 4 may be well suited for including such an additional transducer. As shown in FIGS. 5a and 5b, an additional transducer 210 may take the form of a HIFU transducer and be positioned near a back-side of the DGT 30. As shown in FIG. 5a, transducer 210 may take the form of a fixed-focus simple transducer. As shown in FIG. 5B, a high power DGT (e.g., a DGT fabricated for higher power having a less wide bandwidth than an imaging DGT, that may optionally use air-backing for increased efficiency) may be used. It should be recognized that by virtue of the capability of controlling its beam angle, such a DGT may provide greater flexibility in positioning the HIFU insonifying area than the fixed transducer of FIG. 5A. The combination of transducers 30, 210 may allow rapid sequencing of imaging and HIFU —i.e., when the imaging DGT 30 is facing the target volume, it is excited and images the target, and 180° of rotation later, HIFU could be applied to the same target by exciting transducer 210. Of course, other angles and combinations of transducers may also be used. By being part of the same assembly, the imaging and HIFU therapy transducers may be physically and/or operationally locked together. Further, the effect of HIFU on the tissue may advantageously be monitored during insonification.

According to an aspect of the present invention, the additional HIFU transducer may be added without using additional coaxial cable by adding a switch in the imaging assembly that selectively disconnects the imaging DGT 30 from the coaxial cables and connects the HIFU transducer (conventional or DGT) 210 at appropriate points as the system rotates, and vice-a-versa. A signal, for example a DC level on the coaxial cable, may be used to control the switch that operates in conjunction with rotator 320 and responsively to controller 310, shown in FIG. 1. When the portion of the rotation during which the HIFU transducer was active is complete, the switch may be energized to reconnect the imaging transducer. In this way, during every rotation both HIFU and imaging could take place, providing sufficiently simultaneous image guidance for the HIFU procedure to be considered real-time.

As is well-known to those skilled in the art, the resolution and size of the image and HIFU characteristics are dependent on the f# (focal distance divided by diameter of the aperture) of the focusing mechanism (lens, reflector, etc). For a particular aperture, usually determined by anatomical constraints (e.g., the diameter of a blood vessel, or the size of a heart ventricle) the resolution (the inverse of the beam diameter) is inversely proportional to the f# (as in eq. 3 above). The diameter of the imaged area, however, is proportional to the f# (as in eq. 2 above).

As will be understood by those possessing an ordinary skill in the pertinent arts, the total number of image points, i.e., the size of the imaged area divided by the resolvable elements, is proportional to the square of the aperture divided by the wavelength. According to an aspect of the present invention, the size of the imaged area and the resolution can thus be varied by varying the f#. Different f#'s may be desirable and may be effected at different times. To find an area of interest, say plaque on the wall of an artery, a high f# is desirable and may be used so that a large portion of the artery wall may be rapidly surveyed. To see a small area, such as a portion of plaque, a small region of high resolution would be desirable and may be used, calling for a low f#.

Control of f# for HIFU procedures may be similarly realized. I.e., a high f# produces a large area of insonation, applicable for large tumors, while a low f# makes a small HIFU spot that can be generated quickly, e.g., well suited for cutting an aberrant part of a heart's conduction system causing fibrillation.

According to an aspect of the present invention, variable focal length lenses may be utilized to realize a tunable f# for imaging and/or HIFU insonification. Variable focal length lenses may be realized by using elastomeric membranes for the curved surfaces shown in FIGS. 1, 4, 5a and 5B. The curvature of those surfaces may be controlled by controlling the pressure in a low acoustic-velocity fluid filling the lens. The low-velocity fluid may serve the same purpose as glass, which has a slower light velocity, producing refraction at the curved interface with blood. A thin tube carrying the low-acoustic-velocity fluid can be contained in the axial structures 220 (shown in FIGS. 4, 5a and 5b) to change the pressure on, and hence curvature of, the refracting surface. By changing the pressure, the curvature of the surface changes, thereby predictably changing the focal length of the lens. Low-velocity fluids are known, such as perfluorocarbons, e.g., Fluorinert FG-70 made by 3M, that have indices of refraction of over 2 with acoustic impedances close to that of blood. Such materials may be well suited for use with the present invention. Using such a fluid, variable focus lenses can be constructed, as described in detail in the literature (for example, “Variable-focus lens for ultrasound hyperthermia applications”,1990 Ultrasonics Symposium, pages 16 ˜1-1664, IEEE Press, Piscataway, N.J.). By observing the image obtained, the operator can adjust the focal length and field of view by changing the pressure, much as a zoom lens adjusts the field of view and level of detail in optical systems. As previously discussed, curved DGTs can be used with the variable focus lenses; the curved DGT may provide the basic focus and the variable focus lens may act to modify the focal length. Using a flexible piezoelectric material (e.g. PVDF film as is known in the art) as the elastomer whose curvature changes with change in pressure would allow changing the focus of the DGT as well. Changes in focus can be achieved by various combinations of one or more fixed DGTs with variable focus lenses, variable focus DGTs with fixed lenses, and variable focus lenses with variable focus DGTs, for example. All such combinations are contemplated in the present invention as described herein and with reference to the drawings.

Thus, tunable systems that combine the capability of HIFU with that of real-time volumetric imaging for use in image-guided therapy may be provided.

Those of ordinary skill in the art may recognize that many modifications and variations of the present invention may be implemented without departing from the spirit or scope of the invention. Thus, it is intended that the present invention covers the modifications and variations of this invention provided they come within the scope of the appended claims and their equivalents.

1. An ultrasonic imaging and treatment system comprising:
a diffraction grating transducer; and
a signal source electrically coupled to said diffraction grating transducer and operative: in a first mode to provide a wide-band excitation signal to said diffraction grating transducer to operate said diffraction grating transducer in an imaging mode; and in a second mode to provide a narrow-band excitation signal to said diffraction grating transducer to operate said diffraction grating transducer in a high intensity focused ultrasound insonifying mode.

2. The system of claim 1, further comprising a controller operatively coupled to the signal source.

3. The system of claim 1, wherein said controller comprises a processor and a memory, said memory comprising code being operable by said processor to selectively operate said signal source in said first and second modes.

4. The system of claim 3, wherein said code is further operable by said processor to control rotation of said diffraction grating transducer.

5. The system of claim 4, wherein said diffraction grating transducer comprises a lens.

6. The system of claim 5, wherein said lens is a variable focal length lens.

7. The system of claim 4, wherein said diffraction grating transducer is formed on a curved surface to define a curved diffraction grating transducer, said curved diffraction grating transducer thereby producing a lens effect.

8. The system of claim 7, wherein the curved surface comprises a flexible material whose curvature changes in response to a change in pressure for variably focusing the diffraction grating transducer.

9. The system of claim 8, wherein the flexible material is a piezoelectric material.

10. The system of claim 7, further comprising a lens for focusing the output of said curved diffraction grating transducer.

11. The system of claim 10, wherein the lens comprises a variable focal length lens.

12. The system of claim 3, wherein said code is further operable by said processor to sequence said signal source between said first and second modes.

13. The system of claim 3, wherein said code is further operable by said processor to change a position of said high intensity focused ultrasound relative to said diffraction grating transducer.

14. The system of claim 13, wherein said changing position comprises substantially sweeping said position of said high intensity focused ultrasound relative to said diffraction grating transducer along a predetermined path.

15. The system of claim 14, wherein said path is an arc that corresponds to a focal distance of said diffraction grating transducer.

16. The system of claim 14, wherein said path is substantially straight.

17. The system of claim 16, wherein said path substantially corresponds to a radial line originating at said diffraction grating transducer.

18. The system of claim 1, further comprising means for rotating said diffraction grating transducer.

19. An ultrasonic imaging and treatment system comprising:
an imaging diffraction grating transducer;
a high intensity focused ultrasound transducer; and,
a signal source electrically coupled to said imaging and high intensity focused ultrasound transducers, and being operative: in a first mode to provide a wide-band excitation signal to said diffraction grating transducer to operate said diffraction grating transducer in an imaging mode; and, in a second mode to excite said high intensity focused ultrasound transducer.

20. The system of claim 19, wherein said high intensity focused ultrasound transducer comprises a second diffraction grating transducer and said signal source provides a narrow-band excitation signal to said second diffraction grating transducer to operate said second diffraction grating transducer in a high intensity focused ultrasound insonifying mode in said second mode.

21. The system of claim 19, wherein said diffraction grating transducer and high intensity focused ultrasound transducer are physically coupled together.

22. The system of claim 21, wherein diffraction grating transducer and high intensity focused ultrasound transducer are jointly rotatable.

23. The system of claim 22, wherein said diffraction grating transducer and high intensity focused ultrasound transducer are substantially oppositely disposed.

24. A computer program product for use in connection with a processor to excite at least one diffraction grating transducer of an ultrasonic imaging system to substantially simultaneously image and high intensity focused ultrasound treat a target area, said computer program product comprising a computer readable medium having program code embodied thereon, said program code for causing at least one signal source to:
in a first mode, provide a wide-band excitation signal to said at least one diffraction grating transducer to operate said diffraction grating transducer in an imaging mode; and,
in a second mode, to provide a narrow-band excitation signal to said at least one diffraction grating transducer to operate said diffraction grating transducer in a high intensity focused ultrasound insonifying mode.

25. The computer program product of claim 24, further comprising program code controlling excitation frequencies of the at least one diffraction grating transducer according to the rotational angle of the system.
